(19) United States
(12) Patent Application Publication
(10) Pub. No.: US 2005/0013730 A1
(43) Pub. Date: Jan. 20, 2005

(54) Items and their treatment

(75) Inventor: Heather Allen, Solihull (GB)

Correspondence Address: MERCHANT & GOULD PC, P.O. BOX 2903, MINNEAPOLIS, MN, 55402-0903, US

(73) Assignee: THE SECRETARY OF STATE FOR THE HOME DEPARTMENT, Solihull (GB)

(21) Appl. No.: 10/814,048

(22) Filed: Mar. 31, 2004

(30) Foreign Application Priority Data
Jul. 15, 2003 (GB) 0316520.6
Dec. 4, 2003 (GB) 0328135.9

(51) Int. Cl. C12Q 1/68; A01N 1/00

(52) U.S. Cl. 422/34; 435/6

(57) ABSTRACT

The use of ethylene oxide for the purpose of providing an item substantially free of DNA is provided. Methods of treating items to render them substantially DNA free are also provided. A method of verifying the substantially DNA free status of an item is also provided. A method for producing items is also detailed in which one or more of the products are analysed to establish whether those and/or other product have DNA associated with them after treatment.

This invention concerns improvements in and relating to items and their treatment, and particularly to providing substantially DNA free items and/or methods of treatment which provide that status for items.

To date samples for forensic analysis have been collected, stored, prepared and analysed in and using items which have been sterilised. Such items are sterilised by a variety of techniques including exposure to gamma radiation, beta radiation, combinations of radiation, electron beams and chemicals. The purpose in each case is to sterilise the item; that is to say destroy the viability of existing organisms, such as bacteria, and to prevent their reoccurrence, for instance by preventing development of their spores. Sterilisation is beneficial in this respect.

To date, however, no steps have been taken to ensure items are made ready for use in a form which is substantially free of DNA. This is a fundamentally different issue from being sterile.

The present invention has amongst its aims to provide items which are substantially free of DNA. The present invention has amongst its aims to provide methods of treatment which provide items which are substantially free of DNA. The present invention has amongst its aims the verification of the substantially DNA free status of items. The present invention has amongst its aims the provision of methods for producing items which are substantially free of DNA and are verified as such.

According to a first aspect of the invention we provide the use of a material which includes ethylene oxide for the purpose of providing a substantially DNA free item.

The material may consist of ethylene oxide, but is preferably a mixture.

According to a second aspect of the invention we provide an item which is substantially free of DNA.

The item may be substantially DNA free due to treatment involving contacting the item with a material and/or mixture including ethylene oxide.

According to a third aspect of the invention we provide a method of treatment in which an item has a first pre-treatment state in which it has DNA associated with it and has a second post-treatment state in which the item is substantially free of DNA, the treatment involving contacting the item with a material which includes ethylene oxide.

According to a fourth aspect of the invention we provide a method of treatment in which an item has a first pre-treatment state in which it potentially has DNA associated with it and has a second post-treatment state in which the item is substantially free of DNA, the treatment involving contacting the item with a material which includes ethylene oxide.

The third and fourth aspects of the invention may include one or more of the options, possibilities or features set out elsewhere in this document, including the following. The analysis of one or more such treated items to establish the amount of DNA associated with it after treatment preferably occurs before any use of the item.

The method may further include analysis of one or more such treated items to establish the amount of DNA associated with it after treatment. The method may include treating a number of items in a batch, one or more items from a batch being analysed. The method may include treating a number of items in each of a number of batches, at least one of the items being analysed to establish the amount of DNA associated with it after treatment. The treatment may involve contacting the item with a first volume including ethylene oxide. The treatment may further involve contacting the item with a second volume including ethylene oxide. The method may include one, two or more treatment cycles.

The first and/or second and/or third and/or fourth aspects of the invention may include any of the options, possibilities or features set out elsewhere in this application, and particularly from amongst the following.

The material may be a mixture. Preferably the material is a mixture of gases. The material may include one or more inert gases. The material may include at least 10% by weight of ethylene oxide. The ethylene oxide is preferably used as a gas.

A substantially DNA free item may be an item which upon analysis using the technique detailed in WO01/79541, particularly pages 26 to 44 thereof, indicates a particular level of DNA as present. The particular level may be that of DNA allele drop in associated with a DNA clean environment. The particular level may be indicated as no peak being greater than 100 rfu, preferably no peak greater than 50 rfu, more preferably no peak greater than 20 rfu, still more preferably no peak greater than 10 rfu and ideally no discernable peaks at all. An item having such DNA levels may be considered DNA free.

A substantially DNA free item may be an item which upon analysis gives all peaks as being less than 100 rfu (random fluorescence units), more preferably less than 50 rfu, still more preferably less than 20 rfu and ideally less than 10 rfu. An item having such DNA levels may be considered DNA free.

The item may be free of DNA as far as analysis techniques can determine, particularly all publically known analysis techniques at the date of filing of this application. The item may be substantially DNA free in the context of an analysis process in which a reportable result is a relative detection level of >50 rfu.

The item may be a plastics item. The item may be a container, tube, tip, automation plate, swab, swab shaft, piece of equipment, piece of forensic equipment or part of any of these. The item may previously of have had DNA associated with it. The DNA may have been of a level which upon analysis gave a relative detection level which is expected from an item which is contaminated with DNA. The DNA may have been of a level which upon analysis gave a relative detection level of greater than 1000 rfu, potentially greater than 200 rfu and possibly even greater than 400 rfu. The item may previously have been used.

According to a fifth aspect of the invention we provide a method of verifying the substantially DNA free status of an item, the method including providing the item within a package, providing a biological indicator in association with the package, the biological indicator having a first state and a second state, the biological indicator being converted to the second stage by contact with a material including ethylene oxide, the state of the biological indicator being checked to ensure it is in the second state and hence that the item is substantially DNA free before use of the item.

The biological indicator may be provided inside the packaging. Preferably the biological indicator is isolated from the item in terms of DNA transfer from the biological indicator to the item. The biological indicator in the second state is preferably discernable different from the biological indicator in the first state. The difference may be visually discernable. The fifth aspect of the invention may include any of the features, options or possibilities set out elsewhere in this application.

According to a sixth aspect of the invention we provide a method for producing items in the form of one or more first products of the process and one or more second products of the process, the method including a treatment in which the items are contacted with ethylene oxide, one or more of the first products of the process being provided to a user, one or more of the second products of the process being analysed to establish whether the second product has DNA associated with it in the second post-treatment state.

Preferably the treatment takes the items from a first pre-treatment state in which they potentially have DNA associated with them to a second post-treatment state in which the items are intended to be substantially free of DNA.

The analysis of one or more such treated items to establish the amount of DNA associated with it after treatment preferably occurs before any use of the second product of the process.

The first products of the process may be the same as the second products of the process. The may differ only in the use to which they are put after treatment.

Preferably the first products of the process are provided to an end user. The first products of the process may be provided directly to the end user or via one or more intermediaries. The first products of the process may be subjected to one or more further treatment steps and/or manufacturing steps before being provided to the user.

Preferably the user uses one or more of the first products of the process in the collection and/or storage and/or analysis of DNA, particularly DNA considered in the field of forensic science and/or DNA considered by a law enforcement agency.

The method of production may include the use of one or more of the first products of the process in the collection and/or storage and/or analysis of DNA.

The second products of the process may be analysed by the producer of the first products of the process and/or second products of the process. The second products of the process may be analysed by another party on behalf of the producer preferably the second products of the process are analysed using the technique detailed in WO01/79541, particularly pages 26 to 44 thereof. Preferably the analysis indicates the level of DNA present, with an indication below a threshold being accepted as showing no DNA as being associated with the second products of the process. The threshold may be that no peak greater than 100 rfu, preferably no peak greater than 50 rfu, more preferably no peak greater than 20 tfu, still more preferably no peak greater than 10 rfu and ideally no discernable peaks at all, are present. Preferably where the analysis indicates that the level of DNA present is below the threshold accepted as showing no DNA as being associated with the second products of the process, this is accepted as showing that no DNA is associated with the first products of the process.

Preferably the first products of the process and/or the second products of the process are establish as not having DNA associated with it or them when one or more, and preferably all, of the second products of the process give all peaks as being less than 100 rfu (random fluorescence units), more preferably less than 50 rfu, still more preferably less than 20 rfu and ideally less than 10 rfu. Preferably the first products of the process and/or the second products of the process are establish as not having DNA associated with it or them when one or more, and preferably all, of the second products of the process are free of DNA as far as analysis techniques can determine, particularly all publically known analysis techniques at the date of filing of this application and/or priority date of this application.

Various embodiments of the invention will now be described, by way of example only. A large range of instruments, instrument parts, containers and equipment are routinely treated to provide them in sterile form prior to use. This may include treatment of a new item prior to use or an old item prior to reuse. As a result of the sterilisation the biological activity of species, such as bacteria, spores, viruses and the like is stopped. Such treatment is important in medical fields where biological activity could have a detrimental effect on the health of the patient, for instance.

A range of different treatments are available to achieve sterility. Particularly in relation to plastics items the treatments generally involve exposure to gamma radiation, beta radiation, combinations of beta and gamma radiation, electron beams or chemicals. These treatments are known to stop bacterial activity.

Because of the historic association of forensic science with medicine and because of the same or similar items used in the two fields, items for use in forensic science are treated in the same way as for medical items, hence they are also sterilised.

In forensic science, and other situations, very low levels of DNA are routinely collected, stored, handled and analysed. The amount of DNA which needs to be present in a sample to obtain a useful result gets lower and lower with improved technologies. As a result even small levels of contamination are detectable and hence a problem to accurate results. As a result forensic users face a problem in ensuring their equipment is ideal for purpose. Some suppliers of items to the medical and forensic science operators have referred to their items as being sterile and so may be seen as implying their items are free of contamination including DNA. This is not a correct assumption to make. The applicant's have established that whatever level of sterility which is achieved, this does not equate to there being no risk of DNA being present on or associated with items. As a result items may be contaminating samples and detracting from the results obtained, without forensic scientists being aware.

Other suppliers refer to their items as being free from contamination because they are manufactured and packaged by machine. Whilst this may assist in minimising the chances of contamination it certainly does not eliminate it. Contamination introduced to the machine during its manufacture, during repair or maintenance and/or contamination from the consumable materials, such as packaging used may all transfer to the product. Air bound contamination is also possible. Simply seeking to prevent contamination reaching the items is unlikely to be effective.

The present invention seeks to provide substantially DNA free items, provide methods for achieving this and methods for verifying such a status as applying. The invention does so, by establishing a particular technique which is truly capable of rendering an item DNA free, even where contamination of the item has already occurred. Furthermore, the technique is optimised so as to not interfere with subsequent analysis processes important in forensic science.

Firstly the Applicant conducted analysis on a wide range of sterile items to establish whether they could still give rise to significant sources of DNA despite the sterilisation process. Items which were considered contamination free by seeking to isolate them from sources of contamination were also considered. In a large number of cases DNA from the items was found and hence indicated that contamination of the items was an issue whatever approach was taken to their manufacture.

The impact of the particular form of treatment used to achieve sterility was then considered. In respect of gamma radiation, beta radiation, combinations of the two, electron beams and a variety of chemical treatments, whilst sterility was achieved, DNA was found.

One treatment used for sterilisation, exposure to ethylene oxide, was found by the Applicant's investigations to render items substantially DNA free. Whilst ethylene oxide is a chemical known for achieving sterility, there are no previous reports of it being used to render items substantially DNA free. This is a surprising result, particularly given the demonstrated failure of a wide range of other sterilisation techniques, which are equally effective at achieving sterilisation, to provide such an effect.

By way of comparison an impression of the relative impact of treatment using gamma radiation and treatment using ethylene oxide on contamination arising from each of three individuals is provided in Table 1. The figures represent the relative peak heights observed following analysis. Gamma radiation treatment is one of the most popular sterilisation treatments and is widely used at present on items destined for medical and forensic use.

TABLE 1

| | Individual A | Individual B | Individual C |
|---|---|---|---|
| Control - no | 100 | 100 | 100 |
| treatment | | | |
| Gamma Treatment | 100 | 100 | 100 |
| Ethylene Oxide | 10 | 2.5 | 4 |
| Treatment | | | |

A variety of treatment cycle forms can be used. In one example, however, the item to be treated is placed in a sealable chamber, potentially alongside a large number of other items. The chamber is then sealed and ethylene oxide is introduced, potentially under sub-atmospheric pressure. The ethylene oxide may be introduced mixed with one or more other gases, such as an inert gas. Carbon dioxide may be used for this purpose. After the desired treatment time, which varies between item types, the ethylene oxide is removed. This removal stage may involve substantially evacuating the chamber.

After the ethylene oxide gas treatment the item can then be flushed with nitrogen and then with air. Both these gases may be cleaned of DNA prior to use or the packaging may be relied upon to physically exclude any further DNA. Additional steps may be taken to promote full removal of the ethylene oxide once it has performed its role.

Following treatment according to such a method, the DNA present on the item was considered. This basically involved a normal amplification and analysis process. The DNA results indicated a peak height of 100 rfu. This compared with peak heights of 4000 rfu or more for untreated items or items treated according to other sterilisation techniques.

The impact of further treatment cycles was considered. The use of a further treatment cycle with ethylene oxide of the above type reduced the peak height to non-detectable levels. This compares very favourably with a peak height of 50 rfu which is deemed to be a DNA indication in normal analysis, with rfu values below that level being deemed noise. Further cycles of treatment had no discernable effect on the detected levels given the success with two cycles.

As we as establishing a technique which prevents DNA contamination appearing in the analysis results, it is also important to establish a technique which does not detrimentally effect the subsequent analysis. A key stage of this analysis is amplification of the collected sample using PCR. It is important that any treatment does not inhibit the effectiveness of the amplification. In this regard, one and two cycles of ethylene oxide treatment were established as having no or minimal effect on the profiles obtained by analysis. Three cycles did show a reduced level of profile reporting, potentially due to the inhibiting effect of remaining ethylene oxide on the PCR.

To provide optimum treatment to achieve DNA free status and avoid subsequent contamination between that treatment and use, it is desirable for the item to be in gas permeable packaging. This allows good access for the gaseous ethylene oxide to the item to be treated, but maintains protection against recontamination with DNA after treatment. To provide a ready indication to a user that the item is substantially DNA free because of the treatment it has undergone a biological indicator can be used. This is basically a biological species selected to undergo a discernable change upon sterilisation. The change may be one of colour or other property. Treatment sufficient to sterilise is deemed to be treatment sufficient to provide a substantially DNA free item in such a case.

Validation of the treatment as achieving the desired DNA free status is also a key part of any production process. Whilst a biological indicator might confirm that the treatment has occurred, it does not in itself confirm that treatment has achieved its purpose. To do that, there is a need to periodically take one or more treated items and subject them to a determination of the amount of DNA associated with them. If the treatment is fit for purpose this will be below the detection limit in all cases. Such determinations could be made on standard items which would have been intended for use if they have not been selected at random for DNA level determination. Alternatively, specially contaminated items may be introduced as controls on the basis that if the treatment deals with their high level of contamination, then dealing with contamination which should be low at worst (due to careful production) will not be an issue.

1. The use of a material which includes ethylene oxide for the purpose of providing a substantially DNA free item.

2. An item which is substantially free of DNA.

3. An item according to claim 2 which is substantially DNA free due to treatment involving contacting the item with a mixture including ethylene oxide.

4. A method of treatment in which an item has a first pre-treatment state in which it has DNA associated with it and has a second post-treatment state in which the item is substantially free of DNA, the treatment involving contacting the item with a material which includes ethylene oxide.

5. The method of claim 4 which further includes analysis of one or more such treated items to establish the amount of DNA associated with it after treatment.

6. The method of claim 4 in which the treatment involves contacting the item with a first volume including ethylene oxide and with a second volume of ethylene oxide.

7. A method of verifying the substantially DNA free status of an item, the method including providing the item within a package, providing a biological indicator in association with the package, the biological indicator having a first state and a second state, the biological indicator being converted to the second stage by contact with a material including ethylene oxide, the state of the biological indicator being checked to ensure it is in the second state and hence that the item is substantially DNA free before use of the item.

8. The method of claim 7 in which the biological indicator is provided inside the packaging, the biological indicator being isolated from the item in terms of DNA transfer from the biological indicator to the item.

9. The method of claim 7 in which the biological indicator in the second state is discernable different from the biological indicator in the first state.

10. A method for producing items in the form of one or more first products of the process and one or more second products of the process, the method including a treatment in which the items are contacted with ethylene oxide, one or more of the first products of the process being provided to a user, one or more of the second products of the process being analysed to establish whether the second product has DNA associated with it in the second post-treatment state.

11. A method according to claim 10 in which the first products of the process are the same as the second products of the process, and only differ in the use to which they are put after treatment.
